(19) United States
(12) Patent Application Publication
(10) Pub. No.: US 2024/0058472 A1
(43) Pub. Date: Feb. 22, 2024

(54) C60 HISTIDINE CARNOSINE FUMARATES AND USE

(71) Applicant: Sinapu LLC, Monrovia, CA (US)

(72) Inventor: Peter Butzloff, Walnut, CA (US)

(21) Appl. No.: 17/899,106

(22) Filed: Aug. 30, 2022

Related U.S. Application Data
(63) Continuation-in-part of application No. PCT/US2022/040974, filed on Aug. 20, 2022.

(51) Int. Cl. A61K 47/69; A61K 47/54

(52) U.S. Cl. CPC A61K 47/6949; A61K 47/545; A61K 47/542

(57) ABSTRACT

A nanoparticle composed of buckminsterfullerene bonded to histidine, carnosine, and ferrous or gallous divalent metal (M) fumarate to from a molecular composition of C60-HIS-CAR-M-FUM is provided. This composition when complexed with iron is used as a treatment for hyperuricemia and gout to normalize uric acid removal from the blood and treat progressive kidney dysfunction. This composition when complexed with gallium is used to block the uptake of iron in the treatment of cancers and tumors, and by acting as a proton shuttle, primarily through histidine and C60, to help inhibit glycolysis in the Warburg effect. The composition may be administered as an oral solid formulation or as a liquid formulation.

### CROSS-REFERENCE TO RELATED APPLICATIONS

This application is a Continuation of International Application PCT/US2022/040974 filed on Aug. 20, 2022 which is incorporated herein by reference in its entirety.

### BACKGROUND

### Field of the Invention

A buckminsterfullerene derivative is provided for the treatment of hyperuricemia or gout which causes painful joint inflammation and is associated with kidney dysfunction in type two diabetes. The derivative can also be used to inhibit glycolysis in the Warburg effect, being of use as an anti-cancer treatment. The buckminsterfullerene derivative provides adducts of histidine, carnosine, and a coordinated divalent metal cation being either ferrous fumarate or gallous fumarate. The composition may be administered as an oral solid formulation or as a liquid formulation. When formulated with ferrous fumarate the composition functions to treat anemia and normalize uric acid removal from the blood, thereby alleviating the associated inflammation leading to progressive kidney dysfunction and painful swelling of the joints. When formulated with gallous fumarate the composition functions as an anti-proliferative antitumor and anticancer composition useful as a prophylactic and as a therapeutic treatment.

### Description of the Prior Art

The digestion of purines in the kidneys and gastrointestinal tract produces uric acid, which must be removed from the blood. When this process becomes dysregulated, uric acid crystals may precipitate out with salts normally present in the body to deposit as needles of monosodium urate crystals into the spaces between the joints, in cartilage, and may also settle into tendons, causing abrasion and inflammation. The resulting gout or hyperuricemia has been associated with cardiovascular diseases, chronic kidney disease (CKD), metabolic syndrome, diabetes mellitus (especially type 2), thyroid dysfunction, and psoriasis.

Gut bacteria are species of microbes that live inside our intestines, called enterocytes, and these organisms produce xanthine oxidase to convert purines in food we eat to produce uric acid as part of the digestion process. Many state-of-the-art medications and treatments target and inhibit the xanthine oxidase where it is produced by enterocytes at the intestines to prevent the formation of bacterially generated uric acid. This strategy attempts to decrease uric acid production by xanthine oxidase inhibitors (XOI). Induced suppression by XOI drugs of natural digestive function in the gut bacteria is known to have deleterious effects on long-term gut microbiome health. Therefore, other strategies to treat gout and hyperuricemia are being used or are being actively developed.

The kidney is normally able to remove about 70 percent of uric acid from the bloodstream, where the balance is removed at the intestines where it is produced, by means of a class of substances known as urate transporters. Renal uric acid transporter type 1 (URAT1) is an organic anion transporter that is activated by several commercially available drugs such as lesinurad, probenecid, and benzbromarone to treat gout.

The biology of uric acid transporters is complex and interdependent on various other cycles in the human body. Short term upregulating or downregulating of the activation of some uric acid transporters provide relief of uric acid from the blood serum, but then translates into other effects when the metabolism is further influenced and altered by changes in or progression of comorbid disease pathology, such as type-2 diabetes, that leads to kidney inflammation. Consequently, more drugs are then needed to reduce the inflammation of the kidney. These non-steroidal anti-inflammatory drugs are associated with substantial gastrointestinal, renal, and cardiovascular side effects and cannot reliably address the sources of this inflammation, most of which are unknown. Ultimately, kidney failure results in economically expensive dialysis treatments with unfortunate death as the inevitable result.

One of the causes of gout is inflammation of the joints, especially for diabetes type-2 patients. Oral CAR supplementation has been reported to reduce the development of osteo-arthritis and inflammation resulting from type 2 diabetes. It is also able to decrease reactive oxygen species (ROS) and suppress inflammation by the pro-inflammatory cytokine interleukin (IL)-1-beta, which act on the synovial membranes that lubricate and protect bones in the spaces between joints under high plasma glucose conditions. Supplementation with carnosine or beta-alanine has been reported to reduce fasting glucose and fasting insulin and show some potential to improve glycemic control and confer insulin resistance, however these amino acids are not sufficient or adequate to provide a significant level of protection to improve kidney function or to prevent significant kidney inflammation leading to renal failure.

The medical evidence to date is conclusive that oral CAR supplementation does have anti-glycolysis properties. Carnosine acts by a direct antioxidant mechanism and by bonding to and sequestering reactive carbonyls (RCS), which are the biological byproducts of lipid and glucose oxidation. Glycolysis is the type of energy production favored by cancer cells to proliferate and is characterized by the long-term inability to clear the metabolic system of lactic acid, in which the lactonic state is better known as lactonia. The ability to clear lactic acid and its oxidation product pyruvic acid from mitochondria is enabled by intensive physical exercise, which produces massive amounts of lactic and pyruvic acids in the short term. Thus, repetitive intense exercise enables the enhanced efficiency of lactic acid removal, and the amino acid CAR participates in that removal function in large part because of its proton donation ability. Dysregulated energy use in glycolysis is also a key factor in the progression of type-2 diabetes. Therefore, it is of interest to note that cancers, tumors, and type-2 diabetes each have glycolysis in common because of metabolic disease, and that carnosine is beneficial in the treatment of each of them. Removal of reactive carbonyls by CAR or CAR derivatives may therefore be expected to aid in the treatment of gout, which can be considered a specific downstream effect of the more general metabolic disease.

Less complex uric transport modulation and a more holistic approach to treat the function and reduce the inflammation of the whole kidney is needed. It is believed that a means to adapt to the change of metabolic function and comorbid pathologies affecting whole kidney health as these alter with time and age, is provided by the composition of the present invention.

### SUMMARY

Gout and hyperuricemia are symptoms that will vanish when sufficient kidney function is restored to discharge most uric acid wastes into the urine. This is because 70 percent of all uric acid waste processing is effectively accomplished by the healthy nephrons of functioning kidneys. Carnosine (CAR) and histidine (HIS) have been reported to have properties advantageous to human health, such as providing anti-glycation and antioxidant functions, an ability to maintain pH balance, and to chelate metal cations leading to long term toxicity and oxidative stress, especially from the gradual accumulation of zinc Zn(2+), copper Cu(2+), and lead Pb(2+) cations.

When the composition of the present invention is a C60-CAR-HIS-FeF molecular structure, the two protective amino acids CAR and HIS are bonded to C60 along with a fumarate having dual carboxylic acids to stabilize the bonding of a ferrous or Fe(2+) iron adduct to provide an amoniac ion-exchange function to treat gout and anemia at the kidney where this composition accumulates for eventual metabolic removal.

When the composition of the present invention is a C60-CAR-HIS-GaF molecular structure, the two protective amino acids CAR and HIS are bonded to C60 along with a fumarate having dual carboxylic acids to stabilize the bonding of a gallous or Ga(2+) gallium ion adduct to provide an ion-exchange function to replace iron in the prophylaxis and in the treatment of cancers, especially cancers of the liver and kidney where this composition accumulates for eventual metabolic removal.

In one aspect, the ferrous fumarate derivative of the C60 provides Fe(+2) cations that can exchange with accumulated zinc Zn(2+), copper Cu(2+), and lead Pb(2+) cations that have accumulated in renal nephrons over the course of a lifetime. This releases iron cations into the blood, while acquiring and removing harmful heavy metal cations to detoxify a major source of kidney nephron inflammation and oxidative stress. The C60 functional group provides the site of cation sequestration within the molecular structure that is easily eliminated from the nephrons to be directed into the urine as waste.

In a related aspect, the amino acids histidine and carnosine adducts are bonded to the C60 functional group, in which this composition can synergistically chelate Zn(2+), Cu(2+), and Pb(2+) cations as well as ions of manganese, cobalt, and cadmium, any of which can contribute to nephron inflammation to generate oxidative stress at the kidney. By acting together in proximity, the amino acid adducts are significantly able to detoxify a major source of kidney nephron inflammation while the C60 functional group provides a site of heavy metal cation sequestration that is easily eliminated from the tissues and directed into the urine as waste.

In another aspect, the C60-bonded amino acid functional groups histidine and carnosine of this composition become transiently bonded to the hyaluronic acid functional groups that decorate the collagen fibrils of the nephron tubes. This allows the molecular composition to collect at the location of the kidney structures where inflammation is located, where it assists with the ion-exchange of uric acid for eventual metabolic removal.

In a related aspect, the C60-bonded amino acid functional groups histidine and carnosine provide anti-glycation functions and anti-crosslinking properties as the result of the action of its antioxidant effects, which is the ability to block the oxidation of biomolecules.

In a related aspect, the C60-bonded amino acid functional groups histidine and carnosine provide zwitterions that characteristically and immediately can buffer changes in acidity or basicity to maintain the proper pH balance to the nephrons at the kidney, thereby conferring additional relief from inflammation in the environment of the blood plasma.

In a related aspect, the C60-bonded amino acid functional groups of carnosine react with toxic aldehydes. Toxic aldehydes are the result of lipid peroxidation in pathologies such as type-2 diabetes. Example toxic aldehydes include 4-hydroxy-2-nonenal (HNE), acrolein (ACR), 4-hydroxy-2-hexenal (HHE) and malondialdehyde. These toxic aldehydes react with the carnosine bonded to the C60 of the C60-HIS-CAR-Fe-FUM molecular composition to detoxify a major source of kidney nephron inflammation, commonly termed carbonyl stress, in which the sequestered toxic aldehydes are easily eliminated and directed into the urine as waste.

In another aspect, the C60-bonded amino acid functional groups confer a superior ability to clear lactic acid and its oxidation product pyruvic acid from mitochondria that is enabled by the proton shuttling capacity of the C60-HIS-CAR-Ga-FUM molecular composition which is synergistic in the combined activity of each of its functional groups.

In a related aspect, the clearance of long-term resident lactic acid removes the metabolic type of environment conducive to the establishment of sustained glycolysis in the Warburg effect, thereby providing an anti-cancer function of the C60-HIS-CAR-Ga-FUM molecular composition that is superior to that of any of C60, histidine, or carnosine alone.

In a related aspect, the presence of the gallium as the selected metal adduct of the C60-HIS-CAR-Ga molecular composition confers the ability block the absorption of iron by proliferative cancer cells by the preferred absorption of gallium ion Ga⁽²⁺⁾.

### BRIEF DESCRIPTION OF DRAWINGS

FIG. 1 is an illustration of the molecular structures of buckminsterfullerene (C60), histidine, beta-Alanyl-L-histidine (carnosine), and ferrous fumarate.

FIG. 2 is an illustration of the molecular structures of L-carnosine reaction with C60 to form C60-CAR.

FIG. 3 is an illustration of the molecular structures of L-histidine reaction with C60 to form C60-HIS.

FIG. 4 is an illustration of the molecular structures of L-histidine and L-carnosine reaction with C60 to form C60-HIS-CAR.

FIG. 5 is an illustration of the molecular structures of L-histidine, L-carnosine, and a fumarate of ferrous or gallium that is bonded with C60 to form the C60-HIS-CAR-M-FUM composition.

FIG. 6 is an illustration of exemplary methods of synthesis of C60-HIS-CAR-M-FUM suitable for water-based and solid based oral administration.

FIG. 7 is an illustration of experimental FTIR data for ferrous fumarate (Fe-FUM).

FIG. 8 is an illustration of experimental FTIR data for L-Histidine (HIS).

FIG. 9 is an illustration of experimental FTIR data for L-Carnosine (CAR)

FIG. 10 is an illustration of experimental FTIR data for C60-CAR.

FIG. 11 is an illustration of experimental FTIR data for C60-HIS.

FIG. 12 is an illustration of experimental FTIR data for C60-HIS-Fe-FUM.

FIG. 13 is an illustration of experimental FTIR data for C60-HIS-CAR-Fe-FUM.

FIG. 14 is an illustration of C60-HIS-CAR-Fe-FUM accumulated at the inner surfaces of kidney nephrons.

FIG. 15 is an illustration of C50-HIS-CAR-Ga-FUM accumulated at a neoplastic cell to hinder its ability to accept or utilize iron.

Embodiments are described in detail with reference to the related drawings. Additional embodiments, features, and/or advantages will become apparent from the ensuing description or may be learned by practicing the invention. In the FIGURES, which are not drawn to scale, like numerals refer to like features throughout the description. The following description is not to be taken in a limiting sense but is made merely for describing the general principles of the invention.

### DETAILED DESCRIPTION

The following detailed description, taken in conjunction with the accompanying drawings, is merely exemplary in nature and is not intended to limit the described embodiments or the application and uses of the described embodiments. Any implementation described herein as “exemplary” or “illustrative” is not necessarily to be construed as preferred or advantageous over other implementations.

Furthermore, there is no intention to be bound by any expressed or implied theory presented in the preceding technical field, background, brief summary or the following detailed description. It is also understood that the specific devices, systems, methods, and processes illustrated in the attached drawings, and described in the following specification, are simply exemplary embodiments of the inventive concepts defined in the appended claims that there may be variations to the drawings, steps, methods, or processes, depicted therein without departing from the spirit of the invention. All these variations are within the scope of the present invention. Hence, specific structural and functional details disclosed in relation to the exemplary embodiments described herein are not to be interpreted as limiting, but merely as a representative basis for teaching one skilled in the art to variously employ the present embodiments in virtually any appropriate form, and it will be apparent to those skilled in the art that the present invention may be practiced without these specific details.

Various terms used in the following detailed description are provided and included for giving a perspective understanding of the function, operation, and use of the present invention, and such terms are not intended to limit the embodiments, scope, claims, or use of the present invention.

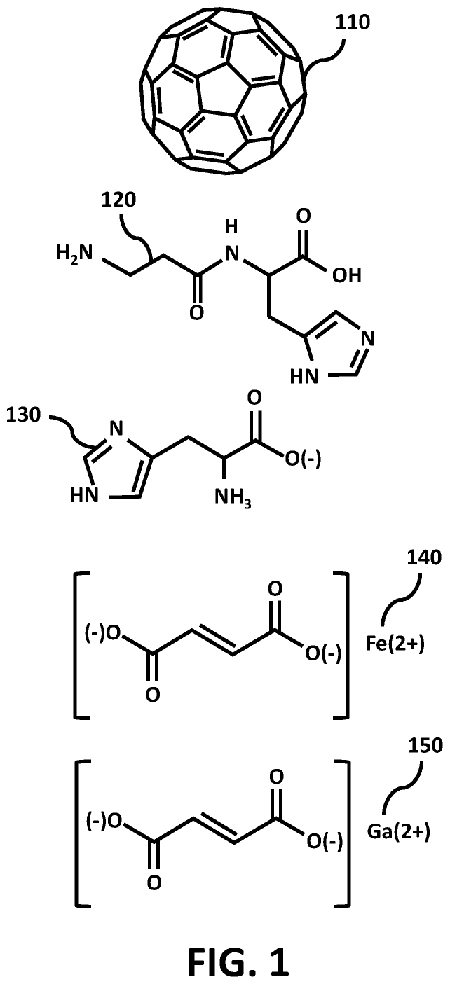

Referring now to the drawings wherein like elements are represented by like numerals throughout, FIG. 1 illustrates the molecular structures used to represent substances used in the composition of the present invention. Buckminsterfullerene 110 is a single molecule comprised of a total of 60 carbon atoms arranged as a sphere and has the chemical formula of C60. L-carnosine (CAR) 120, also known as beta-Alanyl-L-histidine is an amino acid with the chemical formula C₉H₁₄N₄O₃. Carnosine is a well-known anti-inflammatory antioxidant that acts to sequester the reactive carbonyls (RCS) byproducts of lipid and glucose oxidation. Carnosine is also known to stimulate and restore nuclear factor erythroid 2-related factor (nrf2) expression and to induce its nuclear translocation, resulting in improved regulation of the innate immune response in inflammation, and of autophagy, proteostasis, and the physiology of mitochondria. Medical studies have indicated that mitochondrial membrane potential, as an index of the proton (H+) gradient, is typically higher for carnosine treated mitochondria, indicating than a proper homeostasis can be established to ensure a healthy oxidative respiration process in cells. L-histidine (HIS) 130 is a conditionally essential amino acid with the chemical formula C₆H₉N₃O₂. Histidine is a free radical scavenger and chelates divalent metal ions to protect kidney cells from oxidative stress. The antioxidant properties of histidine make it useful to combat excess ferrous ions leading to iron toxicity. Low levels of histidine in the blood plasma have been correlated with an elevated risk of all-cause mortality. Ferrous fumarate (Fe-FUM) 140, also known as iron (II) fumarate is often used as an iron supplement to correct iron deficiency and has the chemical formula C₄H₂FeO₄. Approximately one half of all patients with chronic kidney disease (CKD) are reported to be anemic. When co-administered with histidine to reduce the oxidative stress that is normally caused by iron supplementation, ferrous fumarate has been medically demonstrated to reduce anemia biomarkers and to remediate anemia in patients with low iron plasma that are also experiencing CKD. Gallous fumarate (Ga-FUM) 150, also known as gallium (II) fumarate is used herein as a cancer cell starvation ion to induce an iron deficiency by replacing those biogenic protein sites which require iron cations to rapidly proliferate neoplastic cells, with gallium cations unable to proliferate neoplastic cells, thereby leaving unaffected those healthy cells that do not require a high iron influx. Substances 110, 120, 130, 140, 150 may be used to help create, process, or deliver parts of the C60-HIS-CAR-FUM molecular composition according to these teachings.

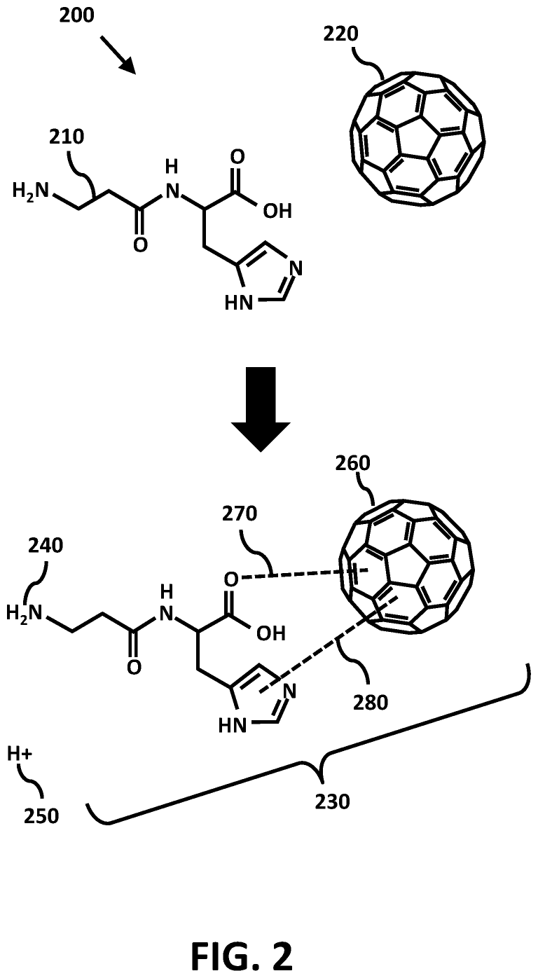

FIG. 2 illustrates a reaction 200 of L-carnosine reaction with C60 to form C60-CAR. As cancer and tumor cells heavily depend on glycolysis for energy generation, L-carnosine (CAR) 210 slows cancer cell growth rates and increases cell death. The glycolytic pathway has been identified in medical studies to be a general target of carnosine. For example, carnosine was medically demonstrated to inhibit the proliferation of human HCT116 colon cancer cells, and in a type of brain cancer termed glioblastoma. Healthy cells that rely on oxidative phosphorylation for respiration metabolism are resistant to L-carnosine's inhibitory growth effects. A similar effect has been documented for buckminsterfullerene (C60) 220, where at least a portion of this benefit arises from anti-oxidant properties. Reactive shear milling can induce the formation of a pi-bonded molecular compound of C60-CAR indicated by bracketed region 230. The terminal amine functional group on CAR 240 can adduct a hydrogen proton 250 to assist with the proton shuttling and antioxidant function in water soluble, hydrophilic cellular environments. The C60 functional group 260 can adduct as many as five hydrogen protons while also promoting their transport through lipid membranes, as the exposed carbon surfaces of the C60 functional group have a hydrophobic, lipophilic characteristic. The stability of the C60-CAR molecular structure is achieved by the pi-carbonyl bond 270, and the pi-pi stacking bond 280 as indicated by the dashed lines in the molecular structure. The enhanced buffering property of C60-CAR with at least one carnosine functional group enables it to enter the mitochondrial matrix, regulate localized enzymes by the control of pH, and promote the oxidative phosphorylation energy metabolism. This material can be used to create part of the C60-HIS-CAR-Fe-FUM molecular composition of the present invention, according to these teachings.

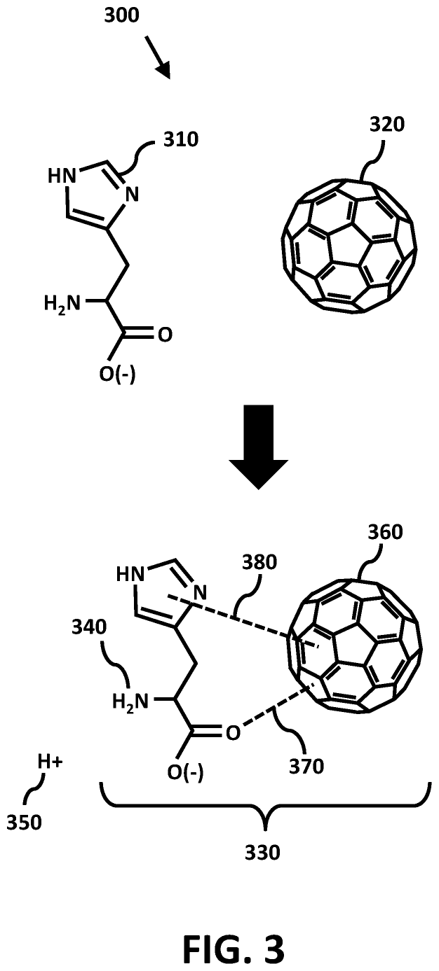

FIG. 3 illustrates a reaction 300 of L-histidine with C60 to form C60-HIS. L-histidine is an antioxidant that can scavenge free radicals and chelate divalent metal ions. As cancer and tumor cells heavily depend on glycolysis for energy generation, L-histidine (HIS) 310 slows cell growth rates and increases cell death. Healthy cells that rely on oxidative phosphorylation for respiration metabolism are resistant to L-histidine's inhibitory growth effects. A similar effect has been documented for buckminsterfullerene (C60) 320, where at least a portion of this benefit arises from anti-oxidant properties. Reactive shear milling can induce the formation of a pi-bonded molecular compound of C60-HIS indicated by bracketed region 330. The terminal amine functional group on HIS 340 can adduct a hydrogen proton 350 to assist with the proton shuttling and antioxidant function in water soluble, hydrophilic cellular environments. The C60 functional group 360 can adduct as many as five hydrogen protons while also promoting their transport through lipid membranes, as the exposed carbon surfaces of the C60 functional group have a hydrophobic, lipophilic characteristic. The stability of the C60-HIS molecular structure is achieved by the pi-carbonyl bond 370, and the pi-pi stacking bond 380 as indicated by the dashed lines in the molecular structure. The enhanced buffering property of C60-HIS with at least one histidine functional group enables it to enter the mitochondrial matrix, regulate localized enzymes by the control of pH, and promote the oxidative phosphorylation energy metabolism. This material can be used to create part of the C60-HIS-CAR-Fe-FUM molecular composition of the present invention, according to these teachings.

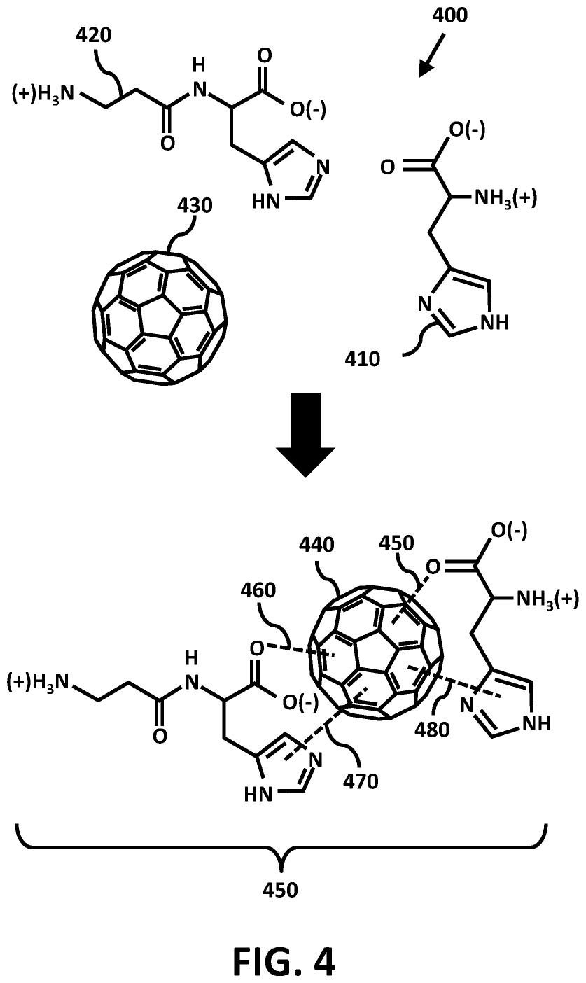

FIG. 4 illustrates a reaction 400 of L-histidine and L-carnosine with C60 to form C60-HIS-CAR 400. L-histidine 410 in combination with L-carnosine 420 are well-known antioxidants that in combination can synergistically scavenge free radicals and chelate divalent metal ions. Carnosine amino acid is in fact created as a derivative of histidine; therefore, it is understood that much of the carnosine functionality arises from the histidine molecular component within its structure. Both carnosine and histidine are normally present as zwitterions at physiological pH, meaning that each of these molecules can have both a positive charged proton adduct and a negative charged deprotonated carboxylic acid group. The C60 molecule 430 reactant molecule is well known to provide free-radical scavenging and anti-oxidant properties. Reactive shear milling can induce the formation of a pi-bonded molecular compound of C60-HIS-CAR indicated by bracketed region 450. The C60 functional group 440 can adduct as many as five hydrogen protons while also promoting their transport through lipid membranes, as the exposed carbon surfaces of the C60 functional group have a hydrophobic, lipophilic characteristic. The stability of the C60-HIS-CAR molecular structure is achieved by the pi-carbonyl bonds 450, 460, and the pi-pi stacking bonds 470, 480 as indicated by the dashed lines in the molecular structure. The enhanced buffering property of C60-HIS-CAR with at least one histidine functional group and at least one carnosine functional group enables it to enter the mitochondrial matrix, regulate localized enzymes by the control of pH, and promote the oxidative phosphorylation energy metabolism. This material is formulated to be a very powerful proton-donating anti-cancer material and can be used to treat gout in those cases where anemia is not a comorbidity. It sufficiently provides a useful capability to protect the kidney nephrons as a utility of the molecular composition of the present invention, according to these teachings.

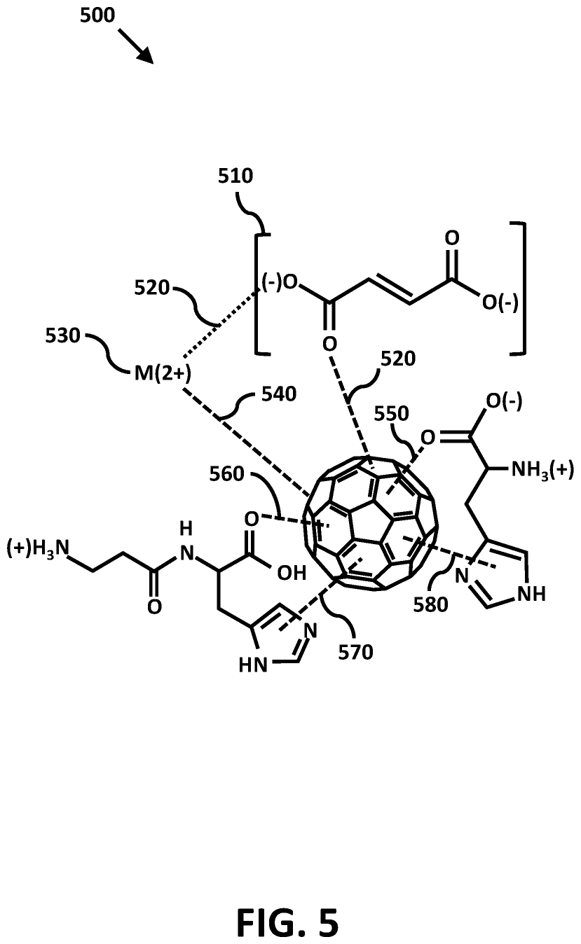

FIG. 5 illustrates the molecular functional groups of L-histidine, L-carnosine, and ferrous fumarate bonded with C60 to form the C60-HIS-CAR-M-FUM molecular composition 500. The fumarate functional group 510 is a dicarboxylic acid having two negative charges at physiological pH. The fumarate functional group 510 has two carbonyls (C═O) within its structure, any of which may form a pi-carbonyl bond 520 with the C60 group. The divalent metal cation M²⁺ 530 is either a ferrous cation Fe²⁺ or a gallous cation Ga²⁺. The positively charged metal cation M²⁺ attracts to and associates with the negatively charged fumarate group 510 as indicated by the dotted line 520 for an ionic type of bond structure, however it also forms a pi-cation bond with the C60 group as indicated by the dashed line 540 in this molecular structure. The further stability of the C60-HIS-CAR-M-FUM molecular structure 500 is achieved by the pi-carbonyl bonds 550, 560, and the pi-pi stacking bonds 570, 580 as indicated by dashed lines. The enhanced buffering property of C60-HIS-CAR-M-FUM 500 with at least one histidine functional group, at least one carnosine functional group, and at least one divalent cationic metal fumarate cation. The fumarate group enables this molecular structure to enter the mitochondrial matrix, regulate localized enzymes by the control of pH.

When co-administered with the amino acid histidine functional group, the amino acid properties mitigate the oxidative properties of the metal cation when the divalent metal cation M²⁺ is formulated as ferrous or Fe²⁺ cation. Ferrous fumarate has been medically demonstrated to reduce anemia biomarkers and to remediate the anemia in patients with low iron plasma, in which an anemia comorbidity is well known to accelerate and worsen the prognosis for chronic kidney disease (CKD). The C60-HIS-CAR-Fe-FUM molecular structure is to be used as a treatment of chronic kidney disease and to functionally address comorbid anemia by use of the C60 group as a targeted delivery vehicle to treat the anemia as one complication of hyperuricemia leading to the conditions of gout and eventual renal failure such as in the more advanced stages of diabetes mellitus. The iron cation Fe²⁺ also functions to promote the oxidative phosphorylation energy metabolism to allow healthy kidney function.

When the divalent metal cation M²⁺ is formulated as the gallous or Ga²⁺ cation, this molecular composition of C60-HIS-CAR-Ga-FUM functions to be a very powerful proton-donating anti-cancer material that is capable of occupying space on cellular proteins which function to displace and prevent the entry or access of free ferrous cations, thereby keeping the iron cations away from proliferating cancer cells.

In summary, the C60-HIS-CAR-M-FUM molecule donates gallium cations when complexed as the C60-HIS-CAR-Ga-FUM complex to contain gallium and provide an anti-proliferative function, and donates iron cations when complexed as the C60-HIS-CAR-Fe-FUM complex to contain iron to treat gout, especially in those cases where anemia is a comorbidity, while protecting the kidney nephrons from oxidative stress and alleviating iron deficiency associated with CKD. The appropriate selection of the iron or the gallium complex used to formulate this molecular structure determines the therapeutic aspect of this composition, according to these teachings.

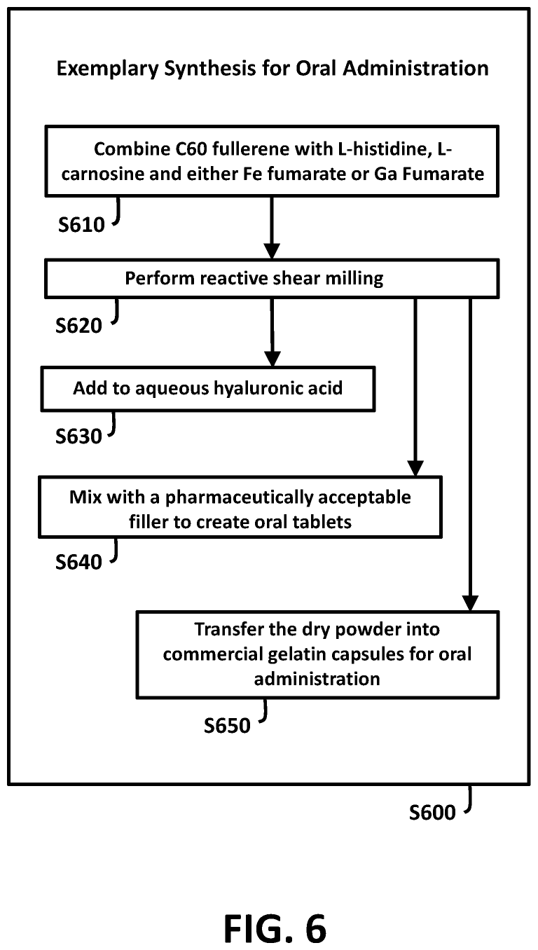

FIG. 6 illustrates the steps of an exemplary method S600 for synthesizing C60-HIS-CAR-FeF suitable for water-based and solid based oral administration. In a step S610 solvent free C60 fullerene is combined with L-histidine in a 3:1 molar ratio of C60 to L-histidine, and also combined with L-carnosine in a 2:1 molar ratio of C60 to L-carnosine, and further combined with ferrous fumarate in about a 1:1 molar ratio of C60 to ferrous fumarate where this ratio may be adjusted to remediate an indicated iron deficiency or an anemia that is medically confirmed to be present in a patient or consumer. In a step S620, reactive shear milling of the dry powder is performed using at least 1000 per second shear rate, below 55° C. for about 25 to about 35 minutes. In a step S630 for some non-limiting embodiments, a desired quantity of the product of step S620 is dissolved into aqueous 0.1% to 0.3% hyaluronic acid. Optionally, colors, flavors, and preservatives such as potassium sorbate or sodium benzoate for beverage servings or liquid administration of oral pharmaceutical solutions are added. In a step S640, for other non-limiting embodiments, the product of step S620 is mixed with a pharmaceutically acceptable solid filler to create oral tablets. Exemplary solid fillers may include calcium stearate, magnesium stearate, calcium phosphate, dried collagen, and natural or synthetic zeolites, and combinations thereof. In a step S650, for still other non-limiting embodiments, the product of step S620 is transferred into commercial gelatin capsules to dispense measured quantities for oral administration.

All the Fourier transform infra-red (FTIR) spectrographs hereinafter were measured by transmittance using the potassium bromide (KBr) compressed flow solid pellet compact preparation method. The material used for analysis was obtained by the method of mixing, crushing, and consolidating under 7 metric tons of pressure, about 0.001 grams of the analyte substance with 1 gram of a diluent solid KBr that is substantially transparent to infrared light, and which flows under pressure to form a translucent pellet of about 0.4 mm thickness. Spectral background subtraction in air using a control pellet of the same mass and thickness having pure KBr was used to obtain a baseline instrument infrared spectral response. This method is generally referred to as the ‘KBr pellet’ sample preparation method, and it is used hereinafter throughout for each FTIR experimental data collection and spectral analysis. The Fourier transform infrared spectrophotometer used herein to obtain FTIR spectra throughout, is a model RF6000 FTIR instrument manufactured by Shimadzu of Japan. Each FTIR data graph hereinafter is provided with a numeric scale ranging from 400 to 4000 to represent reciprocal centimeters or (cm⁻¹) in wavenumbers.

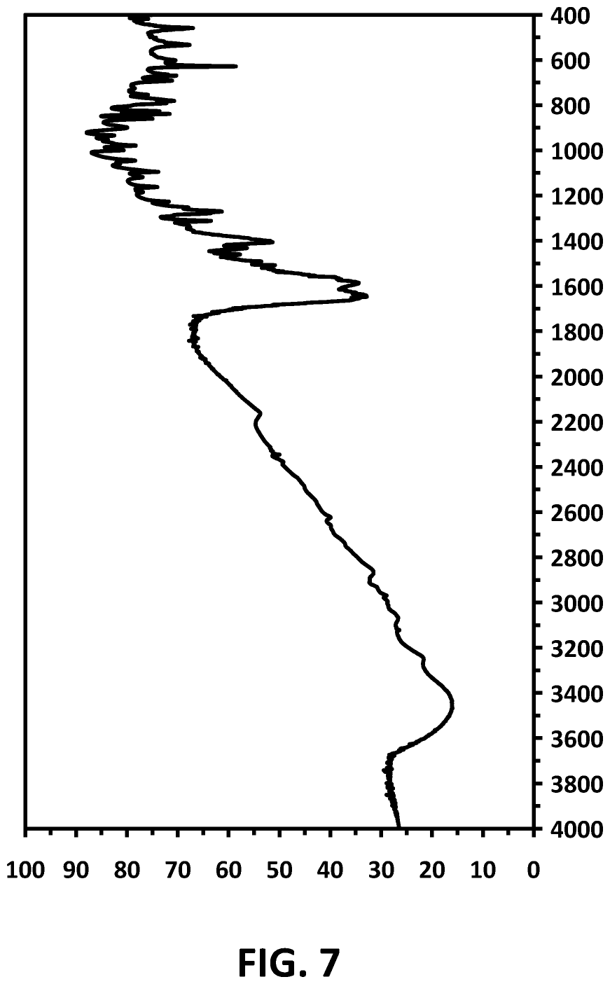

FIG. 7 illustrates the experimental FTIR data for L-carnosine (CAR). The strong and broad FTIR absorbance at 3437 cm⁻¹ is attributed to the presence of the hydroxyl (OH) stretching vibration. The weak absorbance bands at 3252 cm⁻¹ and 3058 cm⁻¹ are attributed to the asymmetric and symmetric stretching of the protonated amine (NH3+) functional group. Two weak carbonyl (C═O) stretching bands appear at 1586 cm⁻¹ and 1642 cm⁻¹. The peak observed at 1406 cm⁻¹ is attributed to a carbon-oxygen-nitrogen (C—O—N) stretching mode. The sharp FTIR absorbances at 669 and 692 cm⁻¹ arise from the neutral imidazole ring of carnosine. The sharp FTIR absorbance at 629 cm⁻¹ arises from the negatively charged and deprotonated carboxylic acid or COO(—) functional group. The overall band absorbances and peak assignments are in accordance with the FTIR spectra reported in published research journal reports for carnosine and serve to help interpret the chemical character of the experimental FTIR results obtained herein.

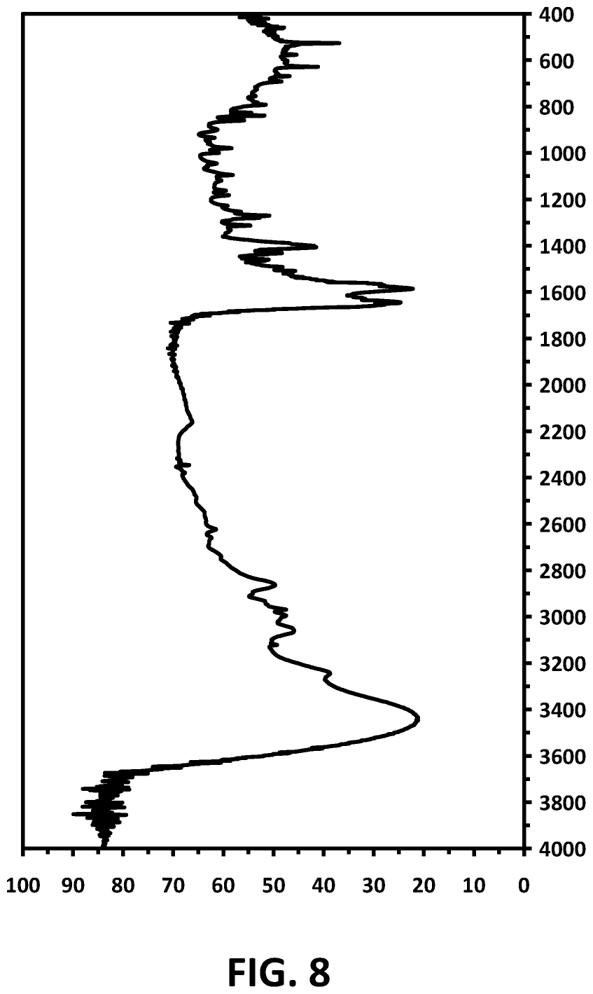

FIG. 8 illustrates the experimental FTIR data for buckminsterfullerene-carnosine (C60-CAR). The strong and broad FTIR absorbance at 3431 cm⁻¹ is attributed to the presence of the hydroxyl (OH) stretching vibration. The weak absorbance bands at 3252 cm⁻¹ and 3056 cm⁻¹ are attributed to the asymmetric and symmetric stretching of the protonated amine (NH3+) functional group of the carnosine adduct. Two carnosine carbonyl (C═O) stretching bands appear at 1586 cm⁻¹ and 1644 cm⁻¹. The peak observed at 1406 cm⁻¹ is attributed to a carbon-oxygen-nitrogen (C—O—N) stretching mode in the carnosine adduct. The weak but sharp FTIR absorbances at 669 cm⁻¹ and 692 cm⁻¹ arise from the neutral imidazole ring of carnosine. The sharp FTIR absorbance at 629 cm⁻¹ arises from the negatively charged and deprotonated carboxylic acid or COO(—) functional group. Two sharp peaks at 576 cm⁻¹ and 526 cm⁻¹ are attributed to the delocalized carbon-carbon aromatic bonds of the C60 functional group. These overall band absorbances and peak assignments provide a chemical characterization of the C60-carnosine molecular ensemble used in these teachings.

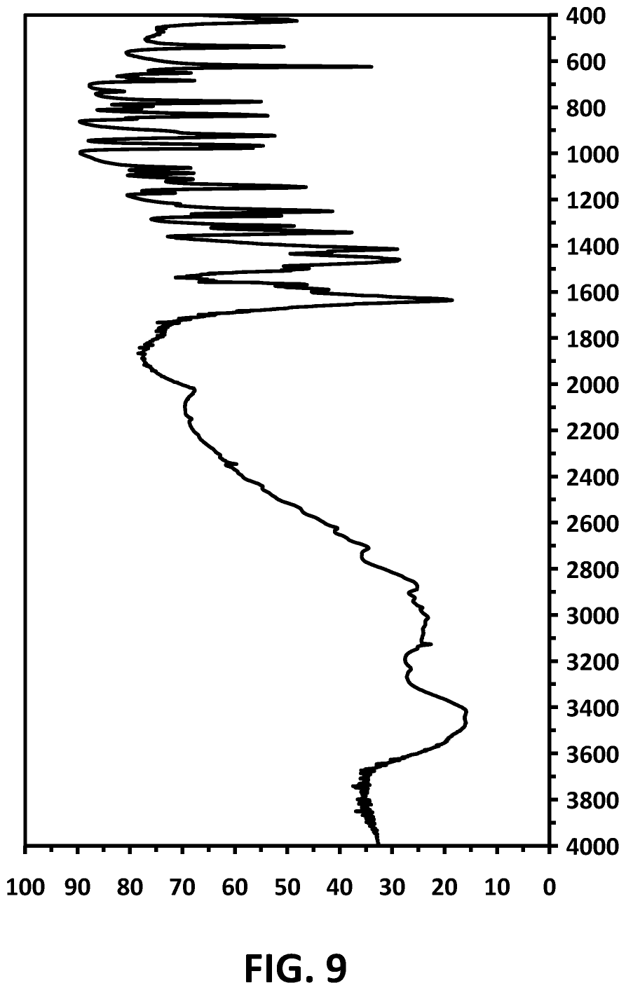

FIG. 9 illustrates the experimental FTIR data for L-histidine (HIS) amino acid. The strong and broad FTIR absorbance at 3467 cm⁻¹ is attributed to the presence of the hydroxyl (OH) stretching vibration. The peak at 1636 cm⁻¹ indicates the carbonyl (C═O) stretching mode of infra-red vibration for histidine. The sharp peak at 1498 cm⁻¹ is attributed to the primary amine (NH₂) bending vibration. CH₂ group deformation vibrations are identified by the sharp band at 1460 cm⁻¹. The carbon-carbon (C—C) stretching mode of vibration occurs at the 1344 cm⁻¹ peak. The weak peak at 1171 cm⁻¹ arises from the carbon-nitrogen (C—N) stretching mode of vibration. The band 1147 cm⁻¹ signifies N—H symmetric bending. C—C—N stretching vibration is observed at 1062 cm⁻¹. The imidazole ring deformation absorbance for histidine occurs at 837 cm⁻¹. The sharp absorbance at 625 cm⁻¹ is attributed to the negatively charged and deprotonated carboxylic acid or COO(—) functional group. The overall band absorbances and peak assignments are in accordance with the FTIR spectra reported in published research journal reports for histidine amino acid and serve to help interpret the chemical character of the experimental FTIR results obtained herein.

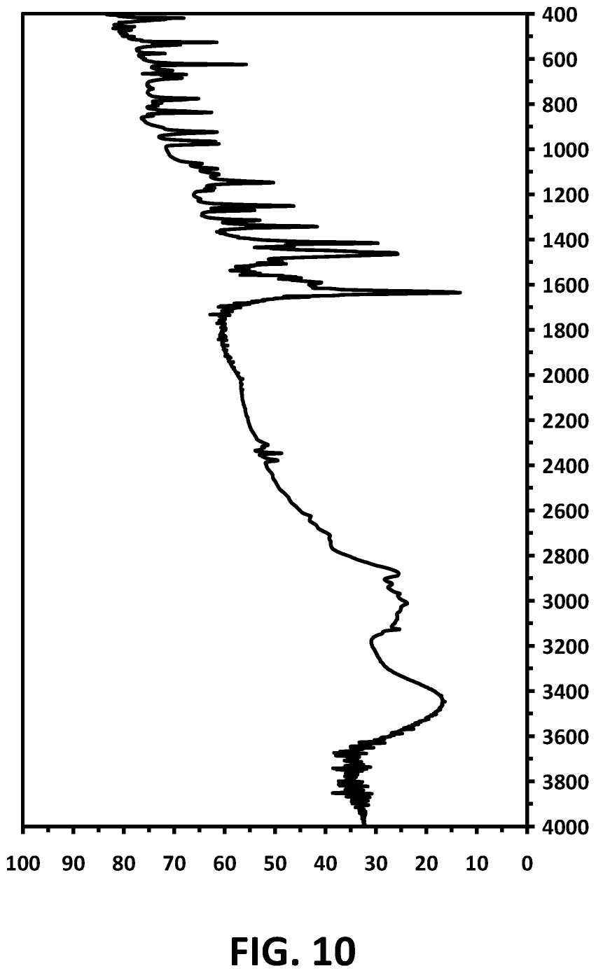

FIG. 10 illustrates the experimental FTIR data for buckminsterfullerene-histidine (C60-HIS). The strong and broad FTIR absorbance at 3447 cm⁻¹ is attributed to the presence of the hydroxyl (OH) stretching vibration. The peak at 1636 cm⁻¹ indicates the carbonyl (C═O) stretching mode of infra-red vibration for histidine. The sharp peak at 1499 cm⁻¹ is attributed to the primary amine (NH₂) bending vibration. CH₂ group deformation vibrations are identified by the sharp band at 1460 cm⁻¹. The carbon-carbon (C—C) stretching mode of vibration occurs at the 1344 cm⁻¹ peak. The band at 1148 cm⁻¹ signifies N—H symmetric bending, which is shifted from the absorbance observed for pure histidine because of the presence of the delocalized C60 group. A weak C—C—N stretching vibration is observed at 1064 cm⁻¹. The imidazole ring deformation absorbance for histidine can be observed at 837 cm⁻¹. The sharp absorbance at 625 cm⁻¹ is attributed to the negatively charged and deprotonated carboxylic acid or COO(—) functional group. These overall band absorbances and peak assignments provide a chemical characterization of the C60-histidine molecular ensemble used in these teachings.

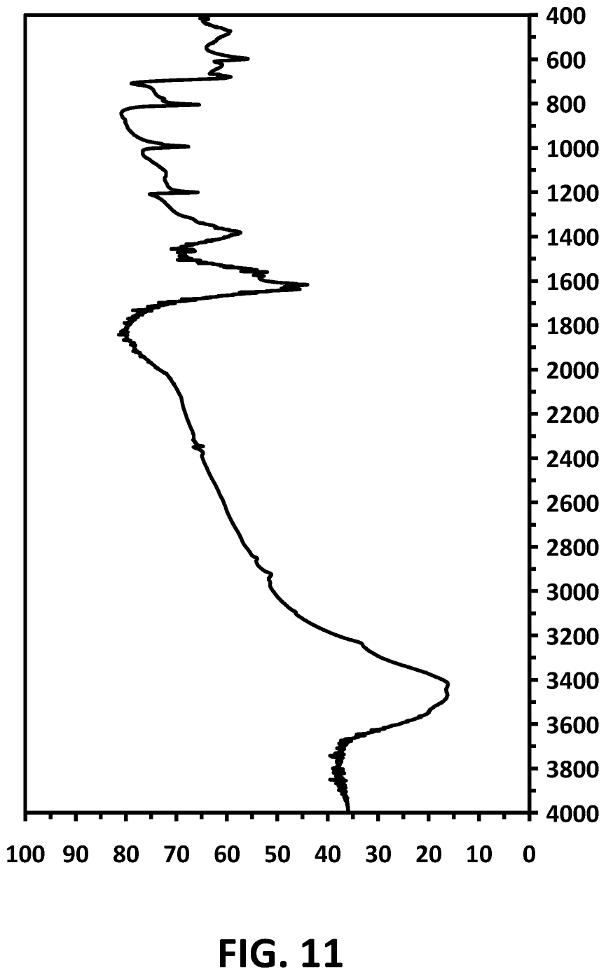

FIG. 11 illustrates the experimental FTIR data for ferrous fumarate (FeF). The broad peak from 3100 cm⁻¹ to 3600 cm⁻¹ centered at 3466 cm⁻¹ arises from the hydroxyl (OH) stretching vibrations. The carbonyl stretching (C═O) of the acid group occurs at 1617 cm⁻¹ for fumarate. This functional group also has in-plane bending vibration modes at 1201 cm⁻¹, 994 cm⁻¹, and 804 cm⁻¹. The carbon-carbon (C—C) stretching modes appear at 1381 cm-1 and 1560 cm⁻¹. These FTIR band absorbances and peak assignments provide a chemical characterization of the commercially available ferrous fumarate material used to create nanoparticle molecular ensembles in these teachings.

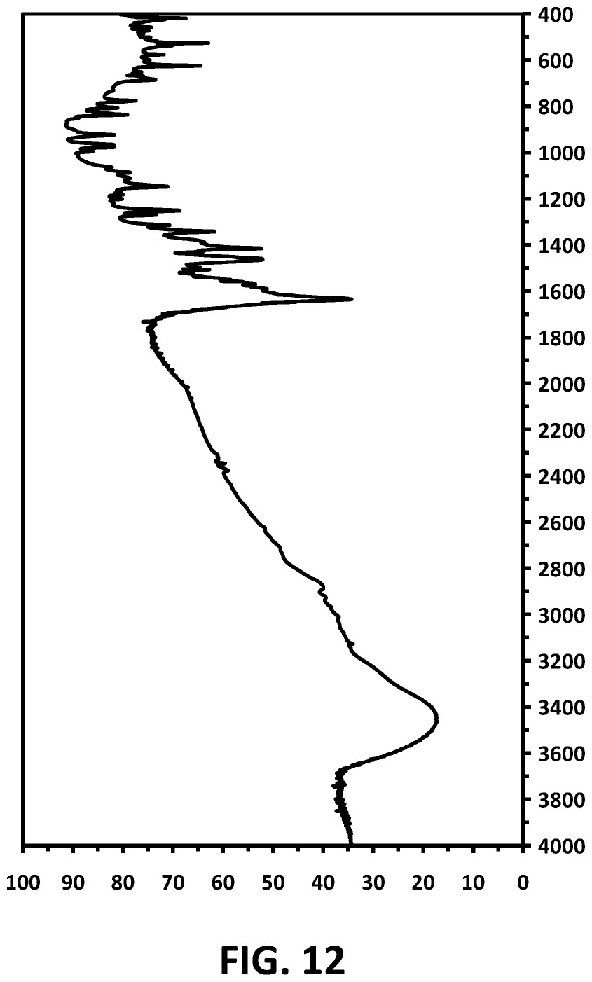

FIG. 12 illustrates the experimental FTIR data for buckminsterfullerene ferrous fumarate histidine or C60-FeF-HIS. The broad peak from 3100 cm⁻¹ to 3600 cm⁻¹ centered at 3436 cm⁻¹ arises from the hydroxyl (OH) stretching vibrations. Interestingly, the wide band of absorbances previously observed for histamine at 2800 cm⁻¹ to 3150 cm⁻¹ are negligible or significantly attenuated, indicating a significant chemical hindrance of the histidine amines to limit their vibration modes in this structure. The peak at 1633 cm⁻¹ is most consistent with the carbonyl (C═O) stretching mode of infra-red vibration for histidine, however it is shifted 3 wavenumbers lower, indicating a somewhat altered chemical environment for this structure. The sharp absorbance bands at 1459 cm⁻¹, 1416 cm⁻¹, and 626 cm⁻¹ were also observed for the C60-HIS derivative as well as for pure histidine. The sharp bands at 526 cm⁻¹ and 624 cm⁻¹ indicate the aromatic C—C vibrations from the C60 functional group. The acid carbonyl in-plane bending mode observed at 806 cm⁻¹ is attributed to a fumarate functional group in which this type of bending vibration was not observed in the histidine or the C60-HIS spectral test results. These FTIR band absorbances and peak assignments provide a chemical characterization consistent with what may be expected for the C60-FeF-HIS nanoparticle ensemble.

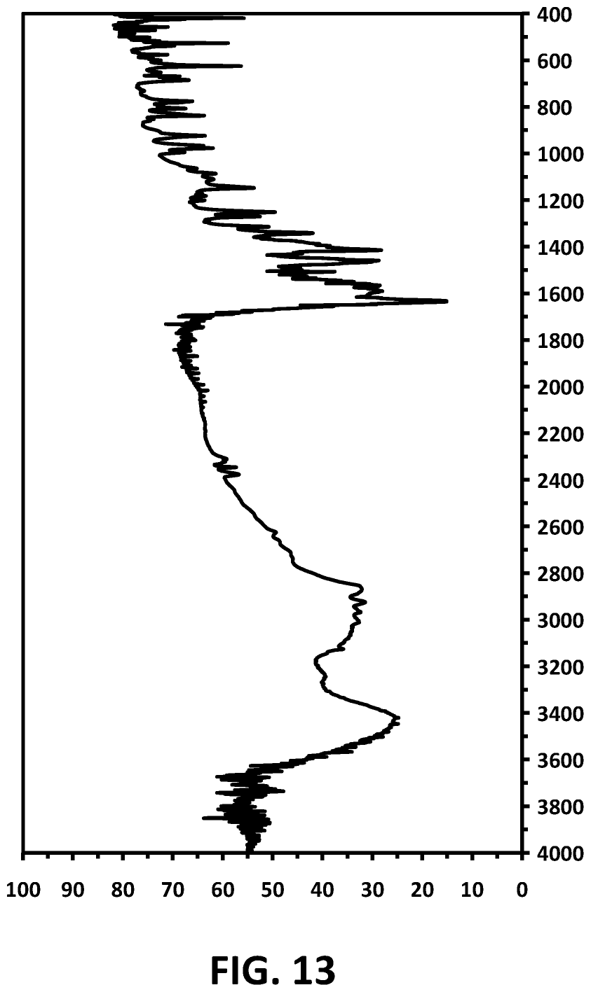

FIG. 13 illustrates the experimental FTIR data for buckminsterfullerene histidine carnosine ferrous fumarate or C60-HIS-CAR-FeF. The wide band of absorbances observed from 2800 cm⁻¹ to 3150 cm⁻¹ are attributed to histidine amines, indicating that these are no longer constrained as was previously observed for the C60-HIS structure. The first carbonyl (C=0) absorbance for carnosine is evident, however the second one previously observed at 1644 cm⁻¹ is now completely absent, which is attributed to a significant interaction of the carnosine acid carbonyl group with the ferrous ion Fe²⁺. A second carbonyl band previously associated with histidine at 1636 cm⁻¹ has shifted significantly to 1632 cm¹, indicating a chemical environment alteration for the acid carbonyl of the histidine group. The sharp bands at 526 cm⁻¹ and 624 cm⁻¹ indicate the aromatic C—C vibrations from the C60 functional group. The complex nature of this FTIR spectrum is unique and is chemically representative of the absorption patterns for the characterization of the nanoparticle molecular ensemble of C60-HIS-CAR-FeF according to these teachings.

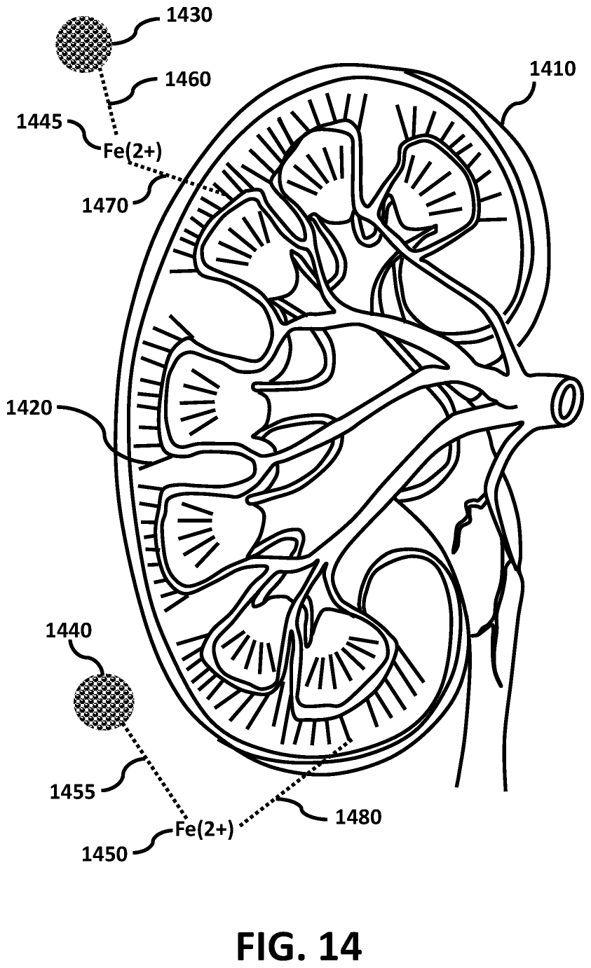

FIG. 14 illustrates C60-HIS-CAR-Fe-FUM accumulated at the inner surfaces of kidney nephrons. One representative kidney 1410 is illustrated in a cut-away view to show the inner organ structures and a multiplicity of nephrons 1420 which filter blood to remove uric acid and waste products from the blood plasma. A multiplicity of C60-HIS-CAR-Fe-FUM molecules are represented by the shaded circular regions 1430, 1440 wherein each such molecular structure contains the presence of at least one ferrous cation 1445, 1450 which is ionically bonded to their respective C60-HIS-CAR-Fe-FUM molecule as indicated by the dotted lines 1455, 1460 and is also ionically bonded to the charged surfaces of the kidney nephrons as indicated by the dotted lines 1470, 1480. The provided Fe⁺² cations of C60-HIS-CAR-Fe-FUM can exchange with accumulated zinc Zn(2+), copper Cu(2+), and lead Pb(2+) cations that have accumulated in the kidney (renal) nephrons while acquiring and removing these metal cations as adducts to detoxify a major source of kidney nephron inflammation and oxidative stress. The presence of the buckminsterfullerene C60 functional group within the molecular composition of C60-HIS-CAR-Fe-FUM acts as a free radical scavenger by combining any two free radicals to neutralize their highly reactive chemical natures, thereby reducing and removing the source of inflammation in the kidneys as these substances are slowly expelled as waste with uric acid. This alleviates the symptoms of chronic kidney disease, especially those associated with diabetes, and substantially allows the proper blood filtering and waste removal functions of the kidney to resume.

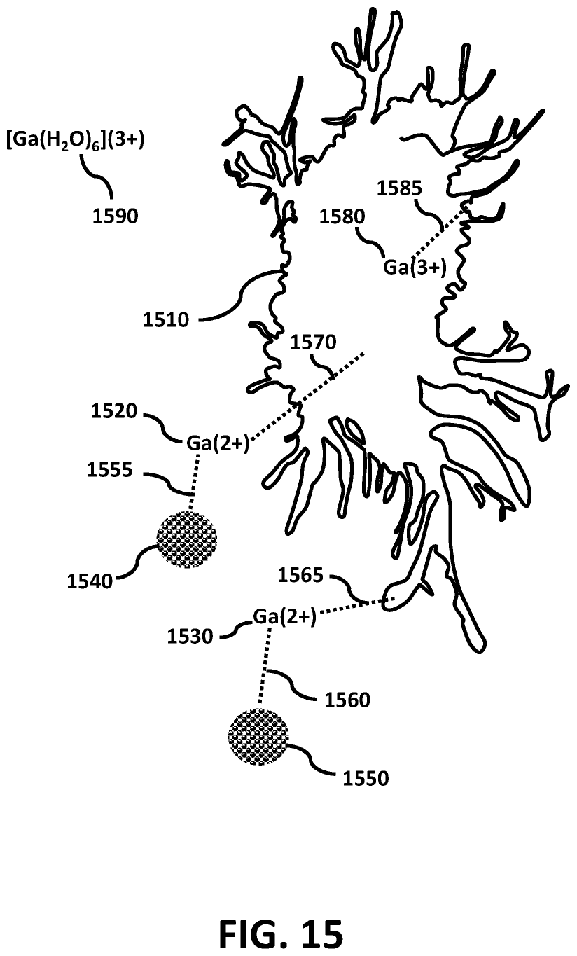

FIG. 15 illustrates C60-HIS-CAR-Ga-FUM accumulated at the inner and outer surfaces of a neoplastic cell such as a cancer cell or a tumor cell. One representative neoplastic cell 1510 requires significant amounts of iron to remain in the state of uncontrolled proliferation, however the protein sites that will normally bind to and sequester the large amounts of iron needed for glycolytic respiration and rapid growth will equally well accept a multiplicity of divalent gallium ions represented by 1520, 1530. Such gallium ions are introduced by a multiplicity of therapeutic C60-HIS-CAR-Ga-FUM molecules 1540, 1550 to which they are complexed by ionic bonds 1555, 1560 as indicated by the dotted lines to the gallous cations, wherein each such gallium ion is also adducted by ionic bonds to the neoplastic cellular proteins as indicated by the dotted lines 1565, 1570. The gallium ions of C60-HIS-CAR-Ga-FUM may at any time be released and become further oxidized to a state of Ga⁺³ wherein such dissolved and free wandering gallic cations may also serve to ionically bond with the neoplastic cell 1585, thereby serving to mask the charged and reactive sites that would otherwise bond ionically with iron. Some of the Ga⁺³ is eventually released to bond ionically with water to form a gallium-water hydroxide 1590 that is removed over time by the liver and kidneys in the course of normal metabolism, according to these teachings.

As variations, combinations and modifications may be made in the construction and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments but defined in accordance with the foregoing claims appended hereto and their equivalents.

What is claimed is:

1. A molecular structure comprising:
a buckminsterfullerene C60 bonded to each of a histidine functional group, a carnosine functional group, and a fumaric acid functional group.

2. The molecular structure of claim 1 wherein the fumaric acid functional group comprises ferrous fumarate.

3. The molecular structure of claim 1 wherein the fumaric acid functional group comprises gallous fumarate.

4. A method of curing, treating, or prophylactically avoiding gout or kidney dysfunction in a subject, comprising:
administering to the subject an effective amount of a composition including a buckminsterfullerene C60 bonded to each of a histidine functional group, a carnosine functional group, and a ferrous fumaric acid functional group.

5. The method of claim 4 wherein the composition is disposed in a pharmaceutically acceptable carrier.

6. The method of claim 5 wherein the molecular composition disposed in the pharmaceutically acceptable carrier comprises a tablet, capsule, pill, powder, granule, or solution.

7. The method of claim 4 wherein administering the molecular composition comprises administration by an intravenous, intramuscular, subcutaneous, intrathecal, intraperitoneal, topical, nasal, or oral route.

8. The method of claim 4 wherein an oral dosage comprises up to about 500 mg of the molecular composition.

9. The method of claim 4 wherein administering the molecular composition comprises intramuscular, intravenous, or subcutaneous administration in an amount of from about 0.1 mg/Kg to about 5 mg/Kg.

10. A method of curing, treating, or prophylactically avoiding gout and progressive kidney disease in type 2 diabetes in a subject, comprising:
administering to the subject an effective amount of a composition including a buckminsterfullerene C60 bonded to each of a histidine functional group, a carnosine functional group, and a gallous fumaric acid functional group.

11. The method of claim 10 wherein the composition is disposed in a pharmaceutically acceptable carrier.

12. The method of claim 11 wherein the molecular composition disposed in the pharmaceutically acceptable carrier comprises a tablet, capsule, pill, powder, granule, or solution.

13. The method of claim 10 wherein administering the molecular composition comprises administration by an intravenous, intramuscular, subcutaneous, intrathecal, intraperitoneal, topical, nasal, or oral route.

14. The method of claim 10 wherein an oral dosage comprises up to about 500 mg of the molecular composition.

15. The method of claim 10 wherein administering the molecular composition comprises intramuscular, intravenous, or subcutaneous administration in an amount of from about 0.1 mg/Kg to about 5 mg/Kg.

16. A method of curing, treating, or prophylactically avoiding neoplastic cell proliferation such as from any tumor or cancer in a subject, comprising:
administering to the subject an effective amount of a composition including a buckminsterfullerene C60 bonded to each of a histidine functional group, a carnosine functional group, and a gallous fumaric acid functional group.

17. The method of claim 16 wherein the composition is disposed in a pharmaceutically acceptable carrier.

18. The method of claim 17 wherein the molecular composition disposed in the pharmaceutically acceptable carrier comprises a tablet, capsule, pill, powder, granule, or solution.

19. The method of claim 16 wherein administering the molecular composition comprises administration by an intravenous, intramuscular, subcutaneous, intrathecal, intraperitoneal, topical, nasal, or oral route.

20. The method of claim 16 wherein an oral dosage comprises up to about 500 mg of the molecular composition.

21. The method of claim 16 wherein administering the molecular composition comprises intramuscular, intravenous, or subcutaneous administration in an amount of from about 0.1 mg/Kg to about 5 mg/Kg.

22. A method of making a molecular composition, the method comprising:
bonding a histidine functional group to a buckminsterfullerene C60;
bonding a carnosine functional group to the buckminsterfullerene C60; and
bonding a fumarate functional group to the buckminsterfullerene C60 wherein the fumarate is complexed with a divalent iron or a divalent gallium cation.

23. The method of claim 22 wherein bonding each of the histidine functional group, the carnosine functional group, and the fumarate functional group to the buckminsterfullerene C60 is performed at no more than 55° C.

24. The method of claim 22 wherein bonding each of the histidine functional group, the carnosine functional group, and the fumarate functional group to the buckminsterfullerene C60 is performed by reactive shear milling.

25. The method of claim 22 wherein bonding the histidine functional group, the carnosine functional group, and the fumarate functional group to the buckminsterfullerene C60 are performed together.

26. The method of claim 22 further comprising combining the molecular composition with a pharmaceutically acceptable carrier.
